SEQUENCE LISTING (6 sequences)

SEQ ID NO: 1 - DNA, 25 bp, Mouse macrophage cell line
tgcagagttccccaactggtacatc

SEQ ID NO: 2 - DNA, 25 bp, Mouse macrophage cell line
gtgctgcctaatgtccccttgaatc

SEQ ID NO: 3 - DNA, 20 bp, Mouse macrophage cell line
cctgtagcccacgtcgtagc

SEQ ID NO: 4 - DNA, 20 bp, Mouse macrophage cell line
ttgacctcagcgctgagttg

SEQ ID NO: 5 - DNA, 20 bp, Mouse macrophage cell line
atcccatcaccatcttccag

SEQ ID NO: 6 - DNA, 20 bp, Mouse macrophage cell line
cctgcttcaccaccttcttg

(19) United States
(12) Patent Application Publication
(10) Pub. No.: US 2013/0142889 A1
(43) Pub. Date: Jun. 6, 2013

(54) COMPOSITION COMPRISING THE EXTRACT OF PHYSALIS ALKEKENGI VAR. FRANCHETI HORT AS AN ACTIVE INGREDIENT FOR PREVENTING AND TREATING INFLAMMATORY DISEASES

(71) Applicants: Hyung Il Kim, Suwon-si (KR); Insop Shim, Gyeonggi-do (KR); Yoon Sang Kim, Seoul (KR)

(72) Inventors: Hyung Il Kim, Suwon-si (KR); Insop Shim, Gyeonggi-do (KR); Yoon Sang Kim, Seoul (KR)

(73) Assignee: Hyung Il KIM, Suwonsi, Gyeonggi-do (KR)

(21) Appl. No.: 13/700,565

(22) PCT Filed: May 27, 2011
(86) PCT No.: PCT/KR2011/003895

§ 371 Date: Nov. 28, 2012

(30) Foreign Application Priority Data
May 28, 2010 (KR) 10-2010-0049925

(51) Int. Cl. A61K 36/81

(52) U.S. Cl. CPC A61K 36/81
USPC 424/725

(57) ABSTRACT

The present invention is related to the inventive compositions comprising the extract of Physalis alkekengi var francheti Hort showing potent anti-inflammatory effect through various experiments, therefore, it can be used as the effective and safe therapeutics or health food for treating and preventing inflammatory diseases

### TECHNICAL FIELD

The present invention relates to a composition comprising the extract of Physalis alkekengi var. francheti Hort as an active ingredient for preventing and treating inflammatory diseases.

### BACKGROUND ART

Inflammation is a barrier response in the body against the various stimuli caused by the contamination with various contaminants such as bacteria, virus etc or tissue injury, which plays an initial protective role in order to limiting the damage to the damaged region or injured region. Mostly, those inflammatory response results in the removal of etiological factors using the components of innate immunity and the induction of specific acquired immunity etc (Hawiger J., Innate Immunity and inflammation: A Transcriptional Paradigm, Immunologic Research, 23, pp. 99-109, 2001).

Inflammatory response is associated with rubor, tumor, calor, dolor etc, which results in consecutive immune responses such as the increase of focal blood flow and decrease of blood velocity according to vasodilation caused by the action of inflammatory mediator, cytokines etc at inflamed area; the increased efflux of plasma components according to increased blood permeability, the increased efflux of immunocyte according to increased adhesive ability of blood endothelial cell to the circulatory immunocyte and increased movement to infected region caused by chemotaxis etc (Gallo R L et al., Biology and Clinical Relevance of naturally occurring antimicrobial peptides, J. Allergy Clin. Immunol., 110, pp. 823-831, 2002; Graeme B et al., Acute Inflammation, American Journal of Pathology, 86(1), pp. 185-274, 1977).

Inflammatory response at infected region initiates the macrophage response against outer germ and inflammatory mediators such as ROS (reactive oxygen species), RNS (Reactive nitrogen species), prostaglandins, leukotriene etc, as well as pro-inflammatory cytokines such as TNF-α, IL-6, IL-1β etc are reported to be involved in inflammatory responses (Renauld J C, New Insights into the role of cytokines in asthma, J. Clin. Pathol., 54, pp. 577-589, 2001; Blake G J et al., Tumor necrosis factor-α, inflammatory biomarkers and atherogenesis, Eur. Heart J., 23, pp. 345-347, 2002). The activation of NF-κB, a transcription factor inducing the gene expression of the inflammatory mediators plays important role in those inflammatory involved response of macrophage. The genes involved in inflammation such as iNOS (inducible nitric oxide synthase), cyclooxygenase (COX-2), TNF-α, IL-6, IL-1β etc in macrophage cell are transcribed by NF-κB.

NO (nitric oxide), a free radical having short half-life, has been reported to be produced during the oxidation reaction of L-arginine with L-citrulline by the catalyzed action of NOS (nitric oxide synthase). It shows various immune-related function in vivo (Moncada S. et al., Nitric oxide: Physiology, Pathology and Phamarcology. Pharmacol. Rev., 43, pp. 109-142, 1991), however the overproduction of NO may show cell-toxicity, which causes to the damage of host cell and tissue as well as may be involved in various pathological syndrome such as atherosclerosis, carcinogenesis etc (Mordan L J, et al., Inhibitors of endogeneous nitrogen oxide formation block the promotion of neoplastic transformation in C3H10T1/2 fibroblasts, Carcinogenesis, 14, pp. 1555-1559, 1993; Lirk P. et al., Inducible nitric oxide synthase, Time for reappraisal, Current. Drug. Targets. Inflamm. Allergy, 1, pp. 89-108, 2002). There exist three kinds of NOS catalyzing NO reproduction and nNOS (neuronal NOS, NOS11) and eNOS (endothelial NOS, NOS3) always express constitutively, while iNOS is an inducible one which is transcribed by only transcription factors, NF-κB activated by LPS (lipopolysaccharide), a bacterial endotoxin.

A transcription factor, NF-κB binding with IκB, a it's inhibitor, exists as an inactivated form within macrophage whereas NF-κB becomes activated through the phosphorylation of IκB by the action of signaling pathway induced when bacterial LPS binds to Toll-like receptor on cell surface, which results in the release and removal of IκB by the action of proteosome's dissolution process. In the result, NF-κB is moved to nucleus to induce the transcription of inflammation-related genes and NF-κB activation is reported to be involved in Akt signaling pathway (Hattori Y. et al, lipopolysaccharide activate Akt in vascular smooth muscle cells resulting in induction of inducible nitric oxide synthase through nuclear factor-kappa B activation, Eur. J. Pharmacol., 481, pp. 153-158, 2003); as well as the signaling pathways of ERK, c-jun and p38-MAPK (Kim S H, et al., Selenium attenuates lipopolysaccharide-induced oxidative stress responses through modulation of p38 MAPK and NF-κB signaling pathways, Exp. Biol. Medicine, pp 565-701, 2004; Robinson M J et al, mitogen-activated protein kinase pathways, Cur. Opi. Cell Biol., 9, pp. 180-186, 1997).

The control of gene expression at the stage of iNOS transcription in connection with inflammation response, is mostly important to determine the duration period and reproduced level of NO. Accordingly, the control mechanism of iNOS expression and enzymatic activity of iNOS has been used as a main target to study novel anti-inflammatory agent for improving or treating chronic inflammatory disease.

The fructus of Physalis alkekengi var francheti Hort, a perennial herb distributed in Korea, has been reported to contain physalin A, B, C, saponins, flavonoids, alkaloids, carotinoids, resin, pectin, tannin, quercetin, caffecic acid, cinnamic acid, ferulic acid., alizarin, rubierythric acid, purpurin, xanthopurpurin, munjistin, pseudopurpurin etc.

However, there has been not reported or disclosed on the anti-inflammatory activity of the extract of Physalis alkekengi var francheti Hort in any of above cited literatures, the disclosures of which are incorporated herein by reference.

Accordingly, the present inventors have confirmed that the extract of Physalis alkekengi var francheti Hort shows potent anti-inflammatory effect through various experiments, i.e., the inhibitory effect on the NO reproduction and the reproduction of pro-inflammatory cytokines such as iNOS, COX-2, TNF-α, IL-6 and IL-1β which is induced by LPS treatment as well as the reducing effect on gene expression of p-IκBα, iNOS and COX-2 protein using by RAW 264.7 cell line, therefore, it can be used as the effective and safe therapeutics or health food for treating and preventing arthritic disease.

### DISCLOSURE OF INVENTION

### Technical Problem

According to one aspect of the present invention, the present invention provides a composition comprising the extract of Physalis alkekengi var francheti Hort for the prevention and treatment of inflammatory disease.

### Solution to Problem

Accordingly, it is an object of the present invention to provide a pharmaceutical composition comprising the extract of Physalis alkekengi var francheti Hort as an active ingredient for treating or preventing inflammatory disease, together with a pharmaceutically acceptable carrier.

It is an another object of the present invention to provide a use of the extract of Physalis alkekengi var francheti Hort for the manufacture of medicament employed for treating or preventing inflammatory disease in human or mammal.

It is the other object of the present invention to provide a method for treating inflammatory disease in human or mammal comprising administering to said mammal an effective amount of the extract of Physalis alkekengi var francheti Hort, together with a pharmaceutically acceptable carrier thereof.

In accordance with one aspect of the present invention, there provided a health functional food comprising the extract of Physalis alkekengi var francheti Hort for the prevention or improvement of inflammatory disease an active ingredient in an amount effective to prevent and improve said disease, together with a sitologically acceptable additive.

The term “extract” disclosed herein comprises the extract prepared by extracting plant material with water, lower alcohols such as methanol, ethanol or the mixtures thereof, preferably water.

The term “Physalis alkekengi var francheti Hort” disclosed herein comprises the fructus, root, leaf and whole body of Physalis alkekengi var francheti Hort, preferably, the fructus of Physalis alkekengi var francheti Hort.

The term “treatment and prevention of inflammatory diseases” disclosed herein is performed by way of inhibiting the NO reproduction and the reproduction of pro-inflammatory cytokines such as iNOS, COX-2, TNF-α, IL-6 and IL-1β as well as reducing gene expression of p-IκBα, iNOS and COX-2 protein.

The term “inflammatory diseases” disclosed herein comprises an inflammatory high fever, tonsillitis, gastritis, colitis, joint arthritis, nephritis, hepatitis, conjunctivitis, atopic dermatitis, rhinitis, hypertension, diabetes, cancer, asthma, urethritis, cystitis, artherosclerosis, allergy disease, rhinitis, asthma, acute pain disease, chronic pain disease, periodontitis, gingivitis, inflammatory intestine disease, gout, myocardiac infarction, congestive heart failure, angina pectoris, stomach ulcer, ischemic stroke, sepsis, burn or pancreatitis, preferably, an inflammatory high fever, tonsillitis, gastritis, colitis, joint arthritis, nephritis, hepatitis, conjunctivitis, atopic dermatitis, more preferably, an inflammatory high fever or tonsillitis, which is occurred by the over-production of NO and the over-reproduction of pro-inflammatory cytokines such as iNOS, COX-2, TNF-α, IL-6 and IL-1β.

The pharmaceutical composition for treating purposed diseases could contain about 0.01 to 95 w/w %, preferably 0.5 to 50 w/w % of inventive extract or compound of present invention based on the total weight of the composition.

Hereinafter, the present invention is described in detail.

An inventive extract of the present invention can be prepared in detail by following procedures.

For example, the fructus of Physalis alkekengi var francheti Hort is dried, sliced and added with 1 to 20-fold, preferably, approximately 1 to 10-fold volume of distilled water, C₁ to C₄ lower alcohols or the mixtures thereof, preferably the distilled water to perform the 1^{st} extraction water at the temperature ranging from 10° C.˜80° C., preferably, room temperature for the period ranging from 6 to 72 hours, preferably, 12 to 36 hours; the solution is subjected to the 2^{nd} extraction with hot water at the temperature ranging from 10° C.˜100° C., preferably, 50° C.˜90° C., for the period ranging from 1 to 20 hours, preferably, 5 to 15 hours with the extraction method by the extraction with hot water, cold water, reflux extraction or ultra-sonication extraction, preferably, extraction with hot water; the extract is collected with filtration, concentrated under reduced pressure and dried to obtain the extract of the present invention.

Accordingly, it is the other object of the present invention to provide a method for preparing the extract of Physalis alkekengi var francheti Hort comprising the steps consisting of; drying, cutting and slicing Physalis alkekengi var francheti Hort and subjecting to the 1^{st} extraction with 1 to 20-fold, preferably, approximately 1 to 10-fold volume of distilled water, C₁ to C₄ lower alcohols or the mixtures thereof, preferably the distilled water to perform water at the temperature ranging from 10° C.˜80° C., preferably, room temperature for the period ranging from 6 to 72 hours, preferably, 12 to 36 hours at the 1^{st} step; subjecting to the 2^{nd} extraction with hot water at the temperature ranging from 10° C.˜100° C., preferably, 50° C.˜90° C., for the period ranging from 1 to 20 hours, preferably, 5 to 15 hours with the extraction method by the extraction with hot water, cold water, reflux extraction or ultra-sonication extraction, preferably, extraction with hot water at 2^{nd} step; filtrating, concentrating under vaccuo and drying to obtain the inventive extract of the present invention.

Also, the above-described procedures may be modified or subjected to further step to fractionate or isolate more potent fractions or compounds by conventional procedure well-known in the art, for example, the procedure disclosed in the literature (Harborne J. B. Phytochemical methods: A guide to modern techniques of plant analysis, 3^{rd} Ed. pp. 6-7, 1998).

Accordingly, the present invention also provided a pharmaceutical composition comprising the extract of Physalis alkekengi var francheti Hort prepared by the above-described preparation method as an active ingredient for treating or preventing inflammatory disease, together with a pharmaceutically acceptable carrier.

The present invention also provided a use of the extract of Physalis alkekengi var francheti Hort prepared by the above-described preparation method for the preparation of therapeutic agent for the treatment and prevention of inflammatory disease in mammal or human.

It is an object of the present invention to provide a method of treating or preventing inflammatory disease in human or mammal, wherein the method comprises administering a therapeutically effective amount of the extract of Physalis alkekengi var francheti Hort prepared by the above-described preparation method, as an effective ingredient, together with a pharmaceutically acceptable carrier thereof.

It is confirmed that the inventive extract of the present invention prepared by above-described method shows potent anti-inflammatory effect through various experiments, i.e., the inhibitory effect on the NO reproduction and the reproduction of pro-inflammatory cytokines such as iNOS, COX-2, TNF-α, IL-6 and IL-1β which is induced by LPS treatment as well as the reducing effect on gene expression of p-IκBα, iNOS and COX-2 protein using by RAW 264.7 cell line.

The inventive composition for treating and preventing inflammatory diseases may comprise the above-described extract as 0.1˜50% by weight based on the total weight of the composition.

The inventive composition may additionally comprise conventional carrier, adjuvants or diluents in accordance with a using method well known in the art. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The composition according to the present invention can be provided as a pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate etc., but are not limited to them. For topical administration, the extract of the present invention can be formulated in the form of ointments and creams.

Pharmaceutical formulations containing present composition may be prepared in any form, such as oral dosage form (powder, tablet, capsule, soft capsule, aqueous medicine, syrup, elixirs pill, powder, sachet, granule) or topical preparation (cream, ointment, lotion, gel, balm, patch, paste, spray solution, aerosol and the like) or injectable preparation (solution, suspension, emulsion).

The composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration and may be chosen by those skilled in the art. However, in order to obtain desirable effects, it is generally recommended to administer at the amount ranging 0.1 to 1000 mg/kg, preferably, 1 to 100 mg/kg by weight/day of the inventive extract of the present invention. The dose may be administered in single or divided into several times per day. In terms of composition, the amount of inventive extract should be present between 0.01 to 50% by weight, preferably 0.5 to 40% by weight based on the total weight of the composition.

The pharmaceutical composition of present invention can be administered to a subject animal such as mammals (rat, mouse, domestic animals or human) via various routes. All modes of administration are contemplated, for example, administration can be made orally, rectally or by intravenous, intramuscular, subcutaneous, intra-cutaneous, intrathecal, epidural or intra-cerebroven tricular injection.

Accordingly, it is the other object of the present invention to provide a functional health food comprising the extract of Physalis alkekengi var francheti Hort as an active ingredient for the improvement and prevention of inflammatory diseases.

The term “a functional health food” defined herein is “the functional food having enhanced functionality such as physical functionality or physiological functionality by adding the extract of the present invention to conventional food to prevent or improve aimed disease in human or mammal”.

It is the other object of the present invention to provide a health care food comprising the extract of Physalis alkekengi var francheti Hort, together with a sitologically acceptable additive for the prevention and alleviation of aimed disease.

The term “a health care food” defined herein is “the food containing inventive extract of the present invention showing no specific intended effect but general intended effect in a small amount of quantity as a form of additive or in a whole amount of quantity as a form of capsule, pill, tablet etc.

The term “a sitologically acceptable additive” defined herein is “any substance the intended use which results or may reasonably be expected to result-directly or indirectly-in its becoming a component or otherwise affecting the characteristics of any food” for example, thickening agent, maturing agent, bleaching agent, sequesterants, humectant, anticaking agent, clarifying agents, curing agent, emulsifier, stabilizer, thickner, bases and acid, foaming agents, nutrients, coloring agent, flavoring agent, sweetner, preservative agent, antioxidant etc, which had been well-known in the art.

If a substance is added to a food for a specific purpose in that food, it is referred to as a direct additive and indirect food additives are those that become part of the food in trace amounts due to its packaging, storage or other handling.

Above described health foods can be contained in food, health beverage, dietary therapy etc and may be used as a form of powder, granule, tablet, chewing tablet, capsule, beverage etc for preventing or improving aimed disease.

Also, inventive extract can be added to food or beverage for prevention and improvement of aimed disease. The amount of inventive extract in food or beverage as a functional health food or health care food may generally range from about 0.01 to 100 w/w % of total weight of food for functional health food composition. In particular, although the preferable amount of inventive extract of the present invention in the functional health food, health care food or special nutrient food may be varied in accordance to the intended purpose of each food, it is preferably used in general to use as a additive in the amount of inventive extract of the present invention ranging from about 0.01 to 5% in food such as noodles and the like, from 40 to 100% in health care food on the ratio of 100% of the food composition.

Providing that the health beverage composition of present invention contains inventive extract as an essential component in the indicated ratio, there is no particular limitation on the other liquid component, wherein the other component can be various deodorant or natural carbohydrate etc such as conventional beverage. Examples of aforementioned natural carbohydrate are monosaccharide such as glucose, fructose etc; disaccharide such as maltose, sucrose etc; conventional sugar such as dextrin, cyclodextrin; and sugar alcohol such as xylitol, and erythritol etc. As the other deodorant than aforementioned ones, natural deodorant such as taumatin, stevia extract such as levaudioside A, glycyrrhizin et al., and synthetic deodorant such as saccharin, aspartam et al., may be useful favorably. The amount of above described natural carbohydrate is generally ranges from about 1 to 20 g, preferably 5 to 12 g in the ratio of 100 ml of present beverage composition.

The other components than aforementioned composition are various nutrients, a vitamin, a mineral or an electrolyte, synthetic flavoring agent, a coloring agent and improving agent in case of cheese, chocolate et al., pectic acid and the salt thereof, alginic acid and the salt thereof, organic acid, protective colloidal adhesive, pH controlling agent, stabilizer, a preservative, glycerin, alcohol, carbonizing agent used in carbonate beverage et al. The other component than aforementioned ones may be fruit juice for preparing natural fruit juice, fruit juice beverage and vegetable beverage, wherein the component can be used independently or in combination. The ratio of the components is not so important but is generally range from about 0 to 20 w/w % per 100 w/w % present composition. Examples of addable food comprising aforementioned extract therein are various food, beverage, gum, vitamin complex, health improving food and the like.

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

It will be apparent to those skilled in the art that various modifications and variations can be made in the compositions, use and preparations of the present invention without departing from the spirit or scope of the invention.

### Advantageous Effects of Invention

The present invention comprising the extract of Physalis alkekengi var francheti Hort shows potent anti-inflammatory effect through various experiments, i.e., the inhibitory effect on the NO reproduction and the reproduction of pro-inflammatory cytokines such as iNOS, COX-2, TNF-α, IL-6 and IL-1β which is induced by LPS treatment as well as the reducing effect on gene expression of p-IκBα, iNOS and COX-2 protein using by RAW 264.7 cell line, therefore, it can be used as the effective and safe therapeutics or health food for treating and preventing inflammatory disease.

The present invention is more specifically explained by the following figures and examples. However, it should be understood that the present invention is not limited to these examples in any manner.

### BRIEF DESCRIPTION OF DRAWINGS

The above and other objects, features and other advantages of the present invention will more clearly understood from the following detailed description taken in conjunction with the accompanying drawings, in which;

FIG. 1 shows the cell cytotoxicity result of the extract of Physalis alkekengi var francheti Hort;

FIG. 2 shows the inhibitory effect of the inventive extract on the gene expression of IL-1β in the inflammatory response using by LPS;

FIG. 3 shows the inhibitory effect of the inventive extract on the gene expression of TNF-α in the inflammatory response using by LPS;

FIG. 4 shows the RT-PCR analysis on the mRNA level of iNOS;

FIG. 5 shows the RT-PCR analysis on the mRNA level of COX-2;

FIG. 6 presents the inhibitory effect of the inventive extract on the LPS-induced corticosterone.

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples. However, it should be understood that the present invention is not limited to these examples in any manner.

### Mode for the Invention

The following Reference Example, Examples and Experimental Examples are intended to further illustrate the present invention without limiting its scope.

### EXAMPLE 1

### Preparation of the Extract of Physalis Alkekengi var Francheti Hort

280 g of dried fructus of Physalis alkekengi var francheti Hort purchased from KCLB (Korean Cell Line Bank; http://cellbank.snu.ac.kr) in Korea, were sliced, dipped into 2.8 L of water at room temperature for 24 hours and the solution was subjected to hot-water extraction for 2 hours with stirring at 90° C., The residue was filtered and the extraction process was repeated four times. The filtrate was collected, concentrated under vaccuo and lyophilized to obtain 23 g of water soluble extract of Physalis alkekengi var francheti Hort (yield: 8.2%, designated as “PA” hereinafter).

### REFERENCE EXAMPLE 1

### Preparation of Reagents

RPMI-1640 medium, DMEM (Dubecco's modified eagle medium), 10% FBS (Fetal bovine serum), penicillin, streptomycin etc were procured from the commercially available company (Welgene Co. Ltd. LS202-02, S-001-04, LM001-01, Korea); Ezcytox reagent from Daeil lab Co. Ltd. (ez 3000, Korea); Rotary evaporator from BUCHI Co. Ltd (R-124, Germany); Freeze dryer from Iishin (FDU-2200, Korea), TRIzol™ from GIBCO BRL Co. Ltd. (MD, USA); ELISA Reader from Anthos (Multiread 400, Austria); Spectrophotometer from GE healthcare Life Science (Ultraspec 2100 pro, Sweden); Primeacript Rtase from TaKaRa Co. Ltd. (Shiga, Japan); PTC-100 Programmable Thermal Controller™ from MJ Research Waltham Co. Ltd. (MA, USA); and ImageMaster TotalLab™ from Amersham Biosciences Co. Ltd. (Piscataway, N.J., USA).

All the reagent used in the experiment were higher grade than analytical grade.

### REFERENCE EXAMPLE 2

### Cell Culture

RAW 264.7 cell line, a mouse macrophage cell, (Korean Cell Line Bank; http://cellbank.snu.ac.kr in Korea) was cultured in High glucose DMEM medium (Dulbecco's Modified eagle Medium) supplemented with 10% FBS and 100 microgram/ml of streptoamycin at 37° C. in humidified 5% CO₂ incubator and used in the experiment.

### EXPERIMENTAL EXAMPLE 1

### Cell Cytotoxicity

To determine the cell cytotoxicity of the extract prepared in Examples, MTT assay was performed according to the method disclosed in the literature (Hwang H J et al., Inhibitory Effect of amygdalin on polysaccharide-inducible TNF-alpha and IL-1 beta mRNA expression and carrageenan-induced rat arthritis. J. Microbiol. Biotechnol., 18(10), pp. 1641-1647, 2008).

RAW 264.7 cell was inoculated into 96-well plate with adjusting cell concentration to 10⁴ cells/well to incubate for 24 hours and adjusted to minimum medium excluding FBS for 4 hours. 0.1 mg/ml and 1 mg/ml of test sample prepared in Example 1 were added thereto for 24 hours. 1 microgram/ml of LPS was added thereto to incubate for 24 hours and 10 microliter of Ezcytox reagent was added to each well to react with each other for 1 hour. After the incubation, resulting absorbance was determined at 450 nm using by ELISA reader (Variokan, Thermo Eletron).

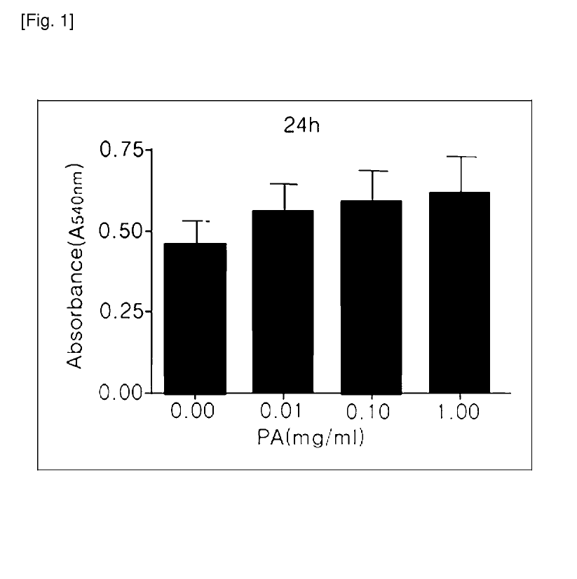

At the result, the group treated with test sample showed significantly increased cell survival rate in a dose dependent manner as can be seen in FIG. 1.

### EXPERIMENTAL EXAMPLE 2

### Effect on Pro-Inflammatory Cytokines

To determine the effect on the gene expression of various cytokines such as IL-1β, TNF-α, iNOS and COX-2 protein using by RAW 264.7 cell line of the extract prepared in Examples, following analysis was performed according to the method disclosed in the literature (Hwang H J et al., Inhibitory Effect of amygdalin on polysaccharide-inducible TNF-alpha and IL-1 beta mRNA expression and carrageenan-induced rat arthritis. J. Microbiol. Biotechnol., 18(10), pp. 1641-1647, 2008).

2-1. Effect on IL-1 Beta Expression

RAW 264.7 cell was inoculated into 96-well plate with adjusting cell concentration to 5×106 cells/well to incubate for 24 hours and adjusted to minimum medium excluding FBS for 4 hours. 0.1 mg/ml and 1 mg/ml of test sample prepared in Example 1 were added thereto for 24 hours. 1 microgram/ml of LPS was added thereto to incubate for 24 hours to be used in following procedure.

The total RNA of the recovered cell was extracted using by TRIzole (GIBCO BRL, MD, USA) and quantitatively determined at 260 nm with spectrophotometer to store at −80° C. After denaturing 1 microgram of total RNA at 65° C. for 10 minutes, reverse transcription reaction was performed in 20 microliter of final reaction mixture using by 0.2 microliter of Primeacript Rtase (TaKaRaq, Shiga, Japan) to obtain cDNA mixture. The cDNA was amplified in 20 microliter of the reaction mixture comprising 10× PCR buffer, each primer and 0.5 U Taq polymerase (TaKaRa, Shiga, Japan) using by PTC-100 Programmable Thermal Controller™ (MJ Research, Walttham, Mass., USA). Each primer was prepared by selecting optimal basic sequences recorded on Genbank and the PCR reaction was performed as shown in Table 1. The amplified DNA was confirmed by electrophoresis in 1.2% agarose gel. The band intensity on the gel was analyzed using by Image Master Total Lab™ (Amersham Biosciences, Piscataway, N.J.) and the quantitative expression level of gene was corrected by using glyceraldehydes 3-phosphate dehydrogenase (GAPDH) as an internal reference.

TABLE 1

| | | | Product | Annealing | |
|---|---|---|---|---|---|
| Primers used in PCR reactions | | | size | temperature | Cycle |
| Genes | | Primer sequence | (bp) | (° C.) | No. |
| IL-1β | Sense | 5′-tgcagagttccccaactggtacatc-3′ | 387 | 60 | 30 |
| | | (SEQ. I.D. 1) | | | |
| | Anti- | 5′-gtgctgcctaatgtccccttgaatc-3′ | | | |
| | sense | (SEQ. I.D. 2) | | | |
| TNF-α | Sense | 5′-cctgtagcccacgtcgtagc-3′ | 374 | 60 | 30 |
| | | (SEQ. I.D. 3) | | | |
| | Anti- | 5′-ttgacctcagcgctgagttg-3′ | | | |
| | sense | (SEQ. I.D. 4) | | | |
| GAPDH | Sense | 5′-atcccatcaccatcttccag-3′ | 579 | 60 | 30 |
| | | (SEQ. I.D. 5) | | | |
| | Anti- | 5′-cctgcttcaccaccacttg-3′ | | | |
| | sense | (SEQ. I.D. 6) | | | |

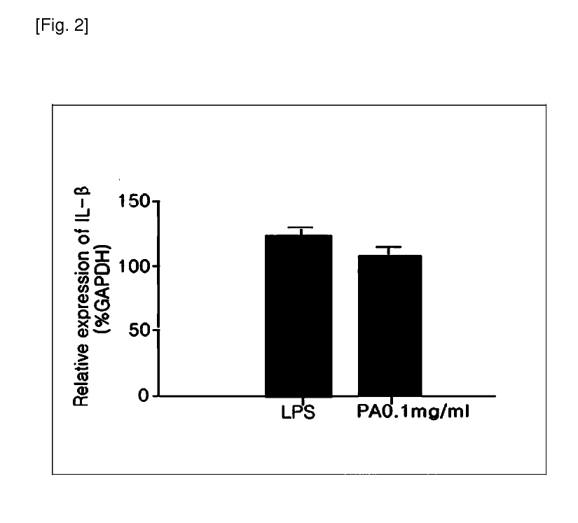

At the result, the expressed mRNA of IL-1β is increased in RAW 264.7 cell treated with LPS but that of IL-1β are significantly decreased in a dose dependent manner in the cell treated with LPS and test sample (See FIG. 2).

2-2. Effect on TNF-α Expression

The effect on the gene expression of TNF-α using by RAW 264.7 cell line of the extract prepared in Examples, was determined according to the similar method disclosed to that in Experimental Example 2-1.

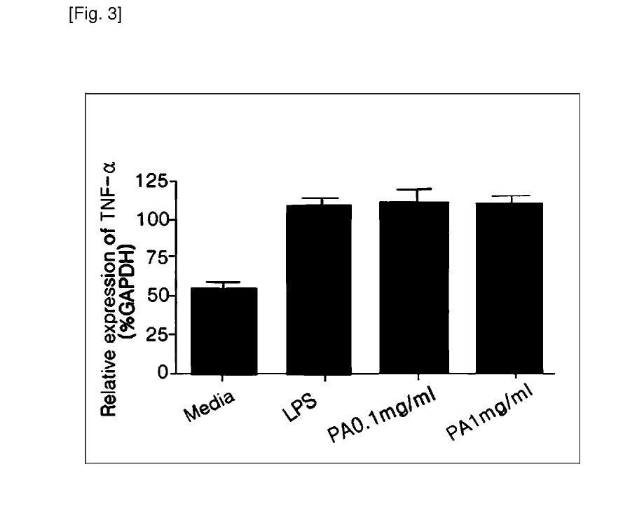

At the result, the expressed mRNA of TNF-α is increased in RAW 264.7 cell treated with LPS but that of TNF-α are significantly decreased in the cell treated with LPS and test sample (See FIG. 3).

2-3. Effect on iNOS Expression

The effect on the gene expression of iNOS using by RAW 264.7 cell line of the extract prepared in Examples, was determined according to the similar method disclosed to that in Experimental Example 2-1.

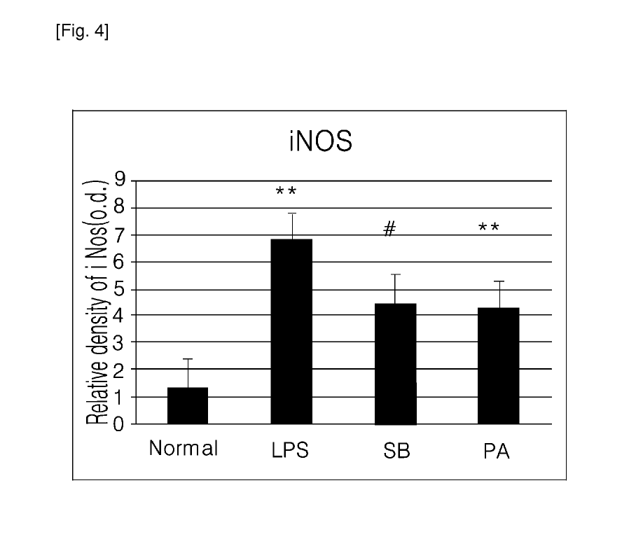

At the result, the expressed mRNA of iNOS is increased in RAW 264.7 cell treated with LPS but that of iNOS are significantly decreased in the cell treated with LPS and test sample (See FIG. 4).

2-4. Effect on the mRNA Expression of COX-2 Enzyme

The effect on the mRNA gene expression of COX-2 enzyme using by RAW 264.7 cell line of the extract prepared in Examples, was determined according to the similar method disclosed to that in Experimental Example 2-1.

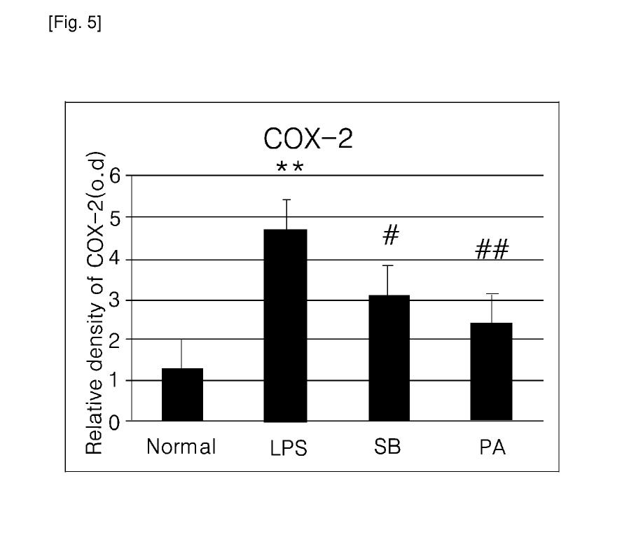

At the result, the expressed mRNA of COX-2 enzyme is increased in RAW 264.7 cell treated with LPS but that of COX-2 enzyme are significantly decreased in the cell treated with LPS and test sample (See FIG. 5).

Accordingly, it has been confirmed that the inhibitory effect of test sample is proved by the decreased level of TNF-α, iNOS and IL-1β, pro-inflammatory factors induced by NF-κB activation.

### EXPERIMENTAL EXAMPLE 3

### Effect on LPS-Induced Corticosterone in Rat Model

To determine the effect on the LPS-induced corticosterone of the extract prepared in Examples, the following assay was performed according to the method disclosed in the literature (Oh J K et al, Antidepressant effects of Soyo-san on Immobilization stress in ovariectomized female rats. Biol. Pharm. Bull., 30(8), pp. 1422-1426, 2007).

The collected blood sample treated with LPS was centrifuged at 4° C. at the speed of 4000 rpm for 15 mins to obtain 50 microliter of blood plasma. The plasma was transferred to test tube, mixed with 5 ml of methylene chloride and left alone for 10 mins at room temperature. The plasma was transferred to another test tube, mixed with 2.5 ml of fluorescent reagent with vortexing for 30 mins and centrifuged at the speed of 2000 rpm for 5 mins to discard the supernatant. The absorbance of residue was determined by spectrofluorometer (excitation: 475 nm, emission: 530 nm) and the determined value was quantitatively determined by comparing with the standard curve made according the various concentration. The mixture with sulfuric acid and ethanol (7:3) was used as a fluorescence reagent.

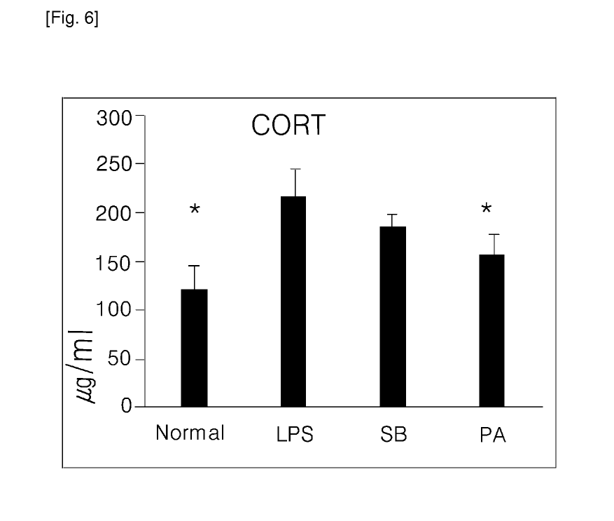

At the result, the test sample (400 mg/kg) potently inhibited the increased corticosterone level comparing with control group treated with LPS only (See FIG. 6).

Hereinafter, the formulating methods and kinds of excipients will be described, but the present invention is not limited to them. The representative preparation examples were described as follows.

Preparation of Injection

PA extract 100 mg

Sodium methabifulfite 3.0 mg

Methyl paraben 0.8 mg

Propyl paraben 0.1 mg

Distilled water for injection optimum amount

Injection preparation was prepared by dissolving active component, controlling pH to about 7.5 and then filling all the components in 2 ml ample and sterilizing by conventional injection preparation method.

Preparation of Powder

PA extract 500 mg

Corn Starch 100 mg

Lactose 100 mg

Talc 10 mg

Powder preparation was prepared by mixing above components and filling sealed package.

Preparation of Tablet

PA extract 200 mg

Corn Starch 100 mg

Lactose 100 mg

Magnesium stearate optimum amount

Tablet preparation was prepared by mixing above components and entabletting.

Preparation of Capsule

PA extract 100 mg

Lactose 50 mg

Corn starch 50 mg

Talc 2 mg

Magnesium stearate optimum amount

Tablet preparation was prepared by mixing above components and filling gelatin capsule by conventional gelatin preparation method.

Preparation of Liquid

PA extract 1000 mg

Sugar 20 g

Polysaccharide 20 g

Lemon flavor 20 g

Liquid preparation was prepared by dissolving active component, and then filling all the components in 1000 ml ample and sterilizing by conventional liquid preparation method.

Preparation of Healthcare Food

PA extract 1000 mg

Vitamin mixture optimum amount

Vitamin A acetate 70 μg

Vitamin E 1.0 mg

Vitamin B1 0.13 mg

Vitamin B2 0.15 mg

Vitamin B6 0.5 mg

Vitamin B12 0.2 μg

Vitamin C 10 mg

Biotin 10 μg

Amide nicotinic acid 1.7 mg

Folic acid 50 μg

Calcium pantothenic acid 0.5 mg

Mineral mixture optimum amount

Ferrous sulfate 1.75 mg

Zinc oxide 0.82 mg

Magnesium carbonate 25.3 mg

Monopotassium phosphate 15 mg

Dicalcium phosphate 55 mg

Potassium citrate 90 mg

Calcium carbonate 100 mg

Magnesium chloride 24.8 mg

The above mentioned vitamin and mineral mixture may be varied in may ways. Such variations are not to be regarded as a departure from the spirit and scope of the present invention.

Preparation of Health Beverage

PA extract 1000 mg

Citric acid 1000 mg

Oligosaccharide 100 g

Apricot concentration 2 g

Taurine 1 g

Distilled water 900 ml

Health beverage preparation was prepared by dissolving active component, mixing, stirred at 85° C. for 1 hour, filtered and then filling all the components in 1000 ml ample and sterilizing by conventional health beverage preparation method.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the inventive composition comprising the extract of Physalis alkekengi var francheti Hort shows potent anti-inflammatory effect through various experiments, therefore, it can be used as the effective and safe therapeutics or health food for treating and preventing inflammatory disease.

Sequence Listing Free Text

| | |
|---|---|
| SEQ. I.D. 1 | 5′-tgcagagaccccaactggtacatc-3′ |
| SEQ. I.D. 2 | 5′-gtgctgcctaatgtccccttgaatc-3′ |
| SEQ. I.D. 3 | 5′-cctgtagcccacgtcgtagc-3′ |
| SEQ. I.D. 4 | 5′-ttgacctcagcgctgagttg-3′ |
| SEQ. I.D. 5 | 5′-atcccatcaccatcttccag-3′ |
| SEQ. I.D. 6 | 5′-cctgcttcaccaccacttg-3′ |

1. A pharmaceutical composition comprising the extract of Physalis alkekengi var francheti Hort as an active ingredient for treating or preventing inflammatory disease, together with a pharmaceutically acceptable carrier.

2. The pharmaceutical composition of claim 1, wherein said extract is prepared by extracting with water, lower alcohols such as methanol, ethanol, or the mixtures thereof.

3. The pharmaceutical composition of claim 1, wherein said Physalis alkekengi var francheti Hort is the fructus, root, leaf or whole body thereof.

4. The pharmaceutical composition of claim 1, wherein said inflammatory diseases are selected from an inflammatory high fever, tonsillitis, gastritis, colitis, joint arthritis, nephritis, hepatitis, conjunctivitis, atopic dermatitis, rhinitis, hypertension, diabetes, cancer, asthma, urethritis, cystitis, artherosclerosis, allergy disease, rhinitis, asthma, acute pain disease, chronic pain disease, periodontitis, gingivitis, inflammatory intestine disease, gout, myocardiac infarction, congestive heart failure, angina pectoris, stomach ulcer, ischemic stroke, sepsis, burn or pancreatitis.

5. A use of the extract of Physalis alkekengi var francheti Hort for the manufacture of medicament employed for treating or preventing inflammatory disease in human or mammal.

6. A method for treating inflammatory disease in human or mammal comprising administering to said mammal an effective amount of the extract of Physalis alkekengi var francheti Hort, together with a pharmaceutically acceptable carrier thereof.

7. A health functional food comprising the extract of Physalis alkekengi var francheti Hort for the prevention or improvement of inflammatory disease an active ingredient in an amount effective to prevent and improve said disease, together with a sitologically acceptable additive.

8. The health care food of claim 7, wherein said health care food is provided as powder, granule, tablet, capsule or beverage type.

9. A method for preparing the extract of Physalis alkekengi var francheti Hort comprising the steps consisting of; drying, cutting and slicing Physalis alkekengi var francheti Hort and subjecting to the 1^{st} extraction with 1 to 20-fold volume of distilled water, C₁ to C₄ lower alcohols or the mixtures thereof to perform water at the temperature ranging from 10° C.˜80° C. for the period ranging from 6 to 72 hours at the 1^{st} step; subjecting to the 2^{nd} extraction with hot water at the temperature ranging from 10° C.˜100° C. for the period ranging from 1 to 20 hours with the extraction method by the extraction with hot water, cold water, reflux extraction or ultra-sonication extraction at 2^{nd} step; filtrating, concentrating under vaccuo and drying step to obtain said extract.
